(19) United States
(12) Patent Application Publication
(10) Pub. No.: US 2024/0279061 A1
(43) Pub. Date: Aug. 22, 2024

(54) HEXAGONAL BORON NITRIDE POWDER AND METHOD FOR PRODUCING SAME, AND COSMETIC PREPARATION AND METHOD FOR PRODUCING SAME

(71) Applicant: Denka Company Limited, Tokyo (JP)

(72) Inventor: Ryuki MATSUI, Tokyo (JP)

(73) Assignee: Denka Company Limited, Tokyo (JP)

(21) Appl. No.: 18/567,533

(22) PCT Filed: Jun. 16, 2021
(86) PCT No.: PCT/JP2021/022905

§ 371 Date: Dec. 6, 2023

(51) Int. Cl. C01B 21/064; A61K 8/19

(52) U.S. Cl. CPC C01B 21/0646; A61K 8/19; A61K 2800/10; A61K 2800/805; C01P 2004/30; C01P 2006/90

(57) ABSTRACT

A hexagonal boron nitride powder, in which an absolute value of a charge amount when 10 g of the hexagonal boron nitride powder is placed in a polyethylene terephthalate container with an inner diameter of 90 mm and a height of 120 mm and stirred at 300 rpm for 5 minutes using a stirring vane 60 mm in diameter with four polytetrafluoroethylene blades is 0.7 nc/g or less.

### TECHNICAL FIELD

The present disclosure relates to a hexagonal boron nitride powder and a method for producing the same, and a cosmetic preparation and a method for producing the same.

### BACKGROUND ART

Boron nitride has lubricity, high thermal conductivity, insulation properties, and the like, and is used in a wide range of applications, including solid lubricants, mold release agents, fillers for resins and rubber, raw materials for a cosmetic preparation (also called a cosmetic), and insulating sintered bodies with heat resistance. Examples of functions of the hexagonal boron nitride powder mixed into the cosmetic preparation include improving slipperiness, spreadability, and concealability of the cosmetic preparation, and imparting glossiness to the cosmetic preparation. In particular, a hexagonal boron nitride powder has superior slipperiness compared to a talc powder and a mica powder, which have similar functions, and is therefore widely used in the cosmetic preparation that requires excellent slipperiness. Patent Literature 1 proposes that the ratio of shear stress to a pressing force be within a predetermined numerical range to improve slipperiness.

Incidentally, static electricity may be generated in a powder due to factors such as friction. Static electricity is thought to affect the properties of the powder. Therefore, as a method for measuring the charge amount of fine particles, there is a known technique of measuring the voltage of a Faraday cage to determine the charge amount using an equation “charge amount=electrostatic capacity×voltage.” Patent Literature 2 proposes a measurement device that can measure the charge amount of powders and granules using a Faraday cage with high sensitivity.

### CITATION LIST

### Patent Literature

[Patent Literature 1] Japanese Unexamined Patent Publication No. 2019-43792
[Patent Literature 2] Japanese Unexamined Patent Publication No. 2010-210385

### SUMMARY OF INVENTION

### Technical Problem

A hexagonal fluorine nitride powder may form agglomerated lumps due to static electricity generated through friction between particles or friction with an inner wall of a container. There is a concern that agglomeration will reduce flowability and impair slipperiness and handleability. Therefore, the present disclosure provides a hexagonal boron nitride powder that can suppress agglomeration due to static electricity, and a method for producing the same. In addition, the present disclosure provides a cosmetic preparation which suppresses agglomeration due to static electricity and has excellent spreadability using the above-described hexagonal boron nitride powder, and a method for producing the same.

### Solution to Problem

According to an aspect of the present disclosure, there is provided a hexagonal boron nitride powder, in which an absolute value of a charge amount when 10 g of the hexagonal boron nitride powder is placed in a polyethylene terephthalate container with an inner diameter of 90 mm and a height of 120 mm and stirred at 300 rpm for 5 minutes using a stirring vane 60 mm in diameter with four polytetrafluoroethylene blades is 0.7 nc/g or less. The hexagonal boron nitride powder may be electrically charged due to, for example, friction between particles and friction with an inner wall of a storage container. Here, if static electricity is generated and the charge amount increases, powder particles will agglomerate due to electrostatic attraction. However, since the above-described hexagonal boron nitride powder has a small absolute value of the charge amount, static electricity generated through friction between particles or friction with an inner wall of a container can be sufficiently suppressed. Accordingly, agglomeration due to static electricity can be suppressed.

In the hexagonal boron nitride powder, the above-described charge amount may be less than −0.1 nc/g. In general, a ceramic powder is likely to agglomerate when charged with static electricity. Since the above-described hexagonal boron nitride powder has a charge amount of less than −0.1 nc/g, agglomeration due to static electricity can be suppressed.

Such a hexagonal boron nitride powder may be used as a raw material for a cosmetic preparation. Since agglomeration of the above-described hexagonal boron nitride powder is suppressed, the hexagonal boron nitride powder has excellent spreadability. Accordingly, it is suitable for use as a raw material for a cosmetic preparation.

A method for producing a hexagonal boron nitride powder according to an aspect of the present disclosure includes: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride; and a firing step of heating and firing a mixed powder containing the calcined product and an aid at 1900° C. to 2100° C. for 10 to 50 hours in an atmosphere of an inert gas, ammonia gas, or a mixed gas thereof.

The above-described production method includes the calcination step of performing firing at a temperature lower than that in the firing step, thereby making it possible to form hexagonal boron nitride with a small particle size and low crystallinity. In the firing step, an aid is used to perform firing at 1900° C. to 2100° C. This increases the crystallinity of hexagonal boron nitride and forms a secondary structure in which primary particles are sterically bound. Thus, a hexagonal boron nitride powder that is less likely to be charged can be obtained. Such a hexagonal boron nitride powder can suppress agglomeration due to static electricity.

In the above-described production method, the fired product obtained in the firing step may be pulverized, washed, and dried to obtain a hexagonal boron nitride powder of which an absolute value of a charge amount when 10 g of the hexagonal boron nitride powder is placed in a polyethylene terephthalate container with an inner diameter of 90 mm and a height of 120 mm and stirred at 300 rpm for 5 minutes using a stirring vane 60 mm in diameter with four polytetrafluoroethylene blades is 0.7 nc/g or less. Since the above-described hexagonal boron nitride powder has a small absolute value of the charge amount, static electricity generated through friction between particles or friction with an inner wall of a container can be sufficiently suppressed. The above-described charge amount may be less than −0.1 nc/g. This makes it possible to suppress agglomeration due to the influence of moisture in atmospheric air.

A cosmetic preparation according to an aspect of the present disclosure includes the above-described hexagonal boron nitride powder. In the above-described hexagonal boron nitride powder, static electricity generated through friction between particles or friction with an inner wall of a storage container can be sufficiently suppressed. Accordingly, agglomeration due to static electricity can be suppressed. The cosmetic preparation containing such a hexagonal boron nitride powder has excellent spreadability.

A method for producing a cosmetic preparation according to an aspect of the present disclosure includes producing the cosmetic preparation using the hexagonal boron nitride powder obtained through any of the above-described production methods as a raw material. The hexagonal boron nitride powder obtained through the above-described production method can suppress agglomeration due to static electricity.

For this reason, the cosmetic preparation produced using such a hexagonal boron nitride powder as a raw material has excellent spreadability.

### Advantageous Effects of Invention

According to the present disclosure, it is possible to provide a hexagonal boron nitride powder that can suppress agglomeration due to static electricity, and a method for producing the same. In addition, according to the present disclosure, it is possible to provide a cosmetic preparation which suppresses agglomeration due to static electricity and has excellent spreadability using the above-described hexagonal boron nitride powder, and a method for producing the same.

### BRIEF DESCRIPTION OF DRAWINGS

FIG. 1 is a view illustrating stirring conditions for a hexagonal boron nitride powder.

FIG. 2 is a bottom view of a stirring vane used for stirring a hexagonal boron nitride powder.

### DESCRIPTION OF EMBODIMENTS

Hereinafter, embodiments of the present disclosure will be described. However, the following embodiments are merely examples for describing the present disclosure and are not intended to limit the present disclosure to the following contents.

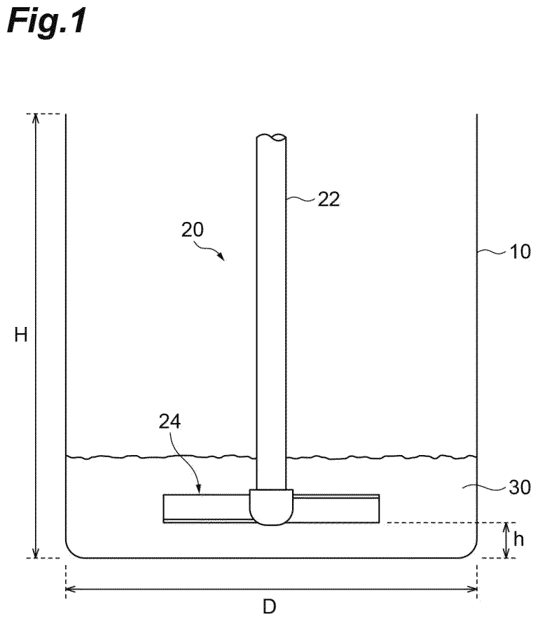

A hexagonal boron nitride powder of the present embodiment has an absolute value of a charge amount of 0.7 nc/g or less when it is stirred at 300 rpm for 5 minutes using a stirring device shown in FIG. 1. Thus, static electricity generated through friction between particles or friction with an inner wall of a container can be sufficiently suppressed during transportation, for example. Accordingly, agglomeration due to static electricity can be suppressed. From the same viewpoint, the absolute value of the above-described charge amount may be 0.6 nc/g or less or 0.5 nc/g or less.

The above-described charge amount may be less than −0.1 nc/g or less than-0.3 nc/g. Since an oxygen atom constituting a water molecule has positive polarity, if the above-described charge amount is a negative value, agglomeration due to the influence of moisture in atmospheric air can be suppressed. An example of a range of the charge amount may be −0.6 to +0.6 nc/g or −0.5 to −0.1 nc/g.

A stirring device 100 of FIG. 1 includes: a bottomed cylindrical polyethylene terephthalate (PET) container 10 having a cylindrical portion and a bottom portion covering one end thereof; a stirrer 20 having a shaft 22 and a stirring vane 24 attached to a distal end thereof; and a rotary motor (not shown in the drawing) on an upper end side of the shaft 22. The rotary motor may be, for example, a three-one motor. The container 10 has an inner diameter D of 90 mm and a height H of 120 mm. At the bottom of the container 10, 10 g of a hexagonal boron nitride powder 30 is stored and deposited in a layered shape. A part of the stirrer 20 is inserted into the container 10 so that the longitudinal direction of the shaft 22 is along the central axis direction of the cylindrical portion. A distance h between the lower end of the stirring vane 24 and the bottom surface of the container 10 is 5 mm.

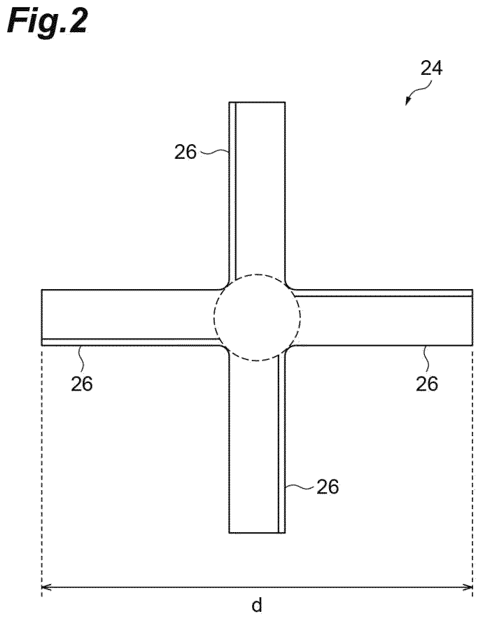

FIG. 2 is a bottom view of the stirring vane 24. The stirring vane 24 attached to a lower end of the shaft has four polytetrafluoroethylene (PTFE) blades 26 that radially extend toward the cylindrical portion of the container 10. A diameter d of the stirring vane 24 is 60 mm. Accordingly, the distance between each distal end of the four blades 26 and the container 10 is 15 mm.

As shown in FIG. 1, the stirrer 20 is started in a state in which the stirring vane 24 is placed in the hexagonal boron nitride powder 30, and the powder is stirred at a rotation frequency of 300 rpm for 5 minutes. After stirring, the charge amount is measured using a commercially available powder friction charge amount measurement device equipped with a Faraday cage. Examples of such measurement devices include NS-K100 (product name) manufactured by Nano Seeds Corporation. A polarity and a magnitude of the charge amount are thought to depend on the surface condition of the hexagonal boron nitride powder. For example, it is thought that as the number of functional groups such as hydroxyl groups increases, charging becomes easier. The charge amount can be adjusted by changing the firing conditions when producing the hexagonal boron nitride powder.

The hexagonal boron nitride according to the present embodiment is less likely to form agglomerated lumps, and therefore has excellent slipperiness and handleability. For this reason, it can be suitably used for various applications. For example, it is used in a releasing agent, an underlay powder, and the like. In addition, this hexagonal boron nitride powder has excellent spreadability when applied to a medium (such as human skin) due to suppressed agglomeration. For this reason, it is suitable for use as a raw material for a cosmetic preparation, for example. That is, the present disclosure can also provide a method of using a hexagonal boron nitride as a raw material for a cosmetic preparation.

A cosmetic preparation according to one embodiment contains the hexagonal boron nitride powder described above. Of the static electricity generated on the surface, this hexagonal boron nitride powder can reduce positive charges more quickly than negative charges. For this reason, agglomeration of the hexagonal boron nitride powder due to moisture is suppressed, and therefore has excellent spreadability.

Examples of a cosmetic preparation include foundation (powder foundation, liquid foundation, and cream foundation), face powder, point makeup, eye shadow, eyeliner, nail polish, lipstick, cheek rouge, and mascara. Among these, the hexagonal boron nitride powders are particularly well suited for foundation and eye shadow. The content of a hexagonal boron nitride powder in a cosmetic preparation is, for example, 0.1 to 70 mass %. A cosmetic preparation can be produced through well-known methods. A method for producing a cosmetic preparation includes, for example, a step of formulating and mixing a hexagonal boron nitride powder with other raw materials.

A method for producing a hexagonal boron nitride powder according to one embodiment includes: a calcination step of firing a raw material powder containing a boron-containing compound powder and a nitrogen-containing compound powder at 600° C. to 1300° C. in an inert gas atmosphere, an ammonia gas atmosphere, or an atmosphere of mixed gas thereof to obtain a calcined product containing at least one selected from the group consisting of low crystalline hexagonal boron nitride and amorphous hexagonal boron nitride; a firing step of heating a mixed powder containing the calcined product and an aid at 1900° C. to 2100° C. for 10 to 50 hours in an atmosphere of an inert gas and/or ammonia gas; and a purification step of pulverizing, washing, and drying the calcined product to obtain a dry powder.

Examples of boron-containing compounds include boric acid, boron oxide, and borax. Examples of nitrogen-containing compounds include cyandiamide, melamine, and urea. The molar ratio between boron atoms and nitrogen atoms in a raw material powder containing boron-containing compound powder and a nitrogen-containing compound powder may be boron atoms:nitrogen atoms=2:8 to 8:2, or 3:7 to 7:3. The raw material powder may contain components other than the above-described compounds. The raw material powder may contain carbonates such as lithium carbonate and sodium carbonate as an aid. In addition, the raw material powder may contain a reducing substance such as carbon.

The raw material powder containing the above-described components is calcined with, for example, an electric furnace in an inert atmosphere such as nitrogen gas, helium gas, or argon gas, an ammonia atmosphere, or a mixed gas atmosphere in which these are mixed. The calcination temperature may be 600° C. to 1300° C., 800° C. to 1200° C., or 900° C. to 1100° C. The calcination time may be, for example, 0.5 to 5 hours or 1 to 4 hours.

The calcined product obtained through calcining contains at least one selected from the group consisting of low crystalline hexagonal boron nitride and amorphous hexagonal boron nitride. In the calcination step, the reaction of boron nitride is allowed to progress at a lower temperature than in the firing step to be described below. For this reason, grain growth can be suppressed, and the particle diameter of a boron nitride powder finally obtained can be reduced. In addition, the specific surface area of the hexagonal boron nitride powder can be increased.

Next, the obtained calcined product is formulated and mixed with an aid to obtain a mixed powder. Examples of aids include borates such as sodium borate and carbonates such as sodium carbonate, calcium carbonate, and lithium carbonate. The formulation amount of the aid may be 2 to 20 parts by mass or 2 to 8 parts by mass based on 100 parts by mass of the calcined product containing hexagonal boron nitride. Such a mixed powder is fired in, for example, an electric furnace in an inert atmosphere such as nitrogen gas, helium gas, or argon gas, an ammonia atmosphere, or a mixed gas atmosphere in which these are mixed.

In the firing step, production and crystallization of boron nitride progress in the presence of the aid. As a result, the crystallinity of the boron nitride contained in the calcined product can be enhanced. The firing temperature is 1900° C. to 2100° C., or may be 1950° C. to 2050° C. The firing time may be, for example, 10 to 50 hours or 15 to 30 hours. By performing firing under such conditions, it is possible to obtain a hexagonal boron nitride powder which has a reduced amount of functional groups (hydroxyl groups) present on the surface of the particles and is less likely to be charged with static electricity.

If the firing temperature becomes too low, the amount of functional groups on the surface of the hexagonal boron nitride tends to increase. If the amount of functional groups in the hexagonal boron nitride increases, the hexagonal boron nitride is likely to be charged with static electricity and tends to easily agglomerate. The same trend is observed even when the firing time is too short. On the other hand, if the firing temperature is too high, the crystal growth of hexagonal boron nitride proceeds too much, whereby primary particles tend to agglomerate. The same trend is observed even when the firing time is too long.

The fired product obtained in the firing step may be pulverized with a usual pulverization device. The pulverized powder may contain impurities in addition to hexagonal boron nitride. Examples of impurities include a residual aid and a water-soluble boron compound. In the purification step, such impurities are reduced through washing. After washing, solid-liquid separation and drying are performed to obtain a dry powder. Examples of washing liquids used for washing include water, an aqueous solution containing an acidic substance, an organic solvent, and a mixed liquid of an organic solvent and water. From the viewpoint of avoiding secondary contamination of impurities, water having an electric conductivity of 1 mS/m or less may be used. Examples of acidic substances include inorganic acids such as hydrochloric acid and nitric acid. Examples of organic solvents include water-soluble organic solvents such as methanol, ethanol, propanol, isopropyl alcohol, and acetone. The washing method is not particularly limited. For example, the pulverized powder may be immersed in a washing liquid and stirred and washed, or the pulverized powder may be washed by spraying a washing liquid.

After the completion of washing, the washing liquid may be subjected to solid-liquid separation through decantation or using a suction filter, a pressure filter, a rotary filter, a sedimentation separator, or a combination thereof. A dry powder may be obtained by drying the separated solid content in a usual dryer. Examples of dryers include a shelf-type dryer, a fluidized bed dryer, a spray dryer, a rotary-type dryer, a belt-type dryer, and a combination thereof. After drying, for example, classification with a sieve may be performed to remove coarse particles.

The above-described hexagonal boron nitride powder can be obtained in this manner. However, it is not always indispensable to perform a purification step. The hexagonal boron nitride powder obtained through the above-described production method has an absolute value of a charge amount of 0.7 nc/g or less when it is stirred at 300 rpm for 5 minutes using the stirring device shown in FIGS. 1 and 2. The absolute value of the above-described charge amount may be 0.6 nc/g or less or 0.5 nc/g or less. The above-described charge amount may be less than −0.1 nc/g or less than −0.3 nc/g from the viewpoint of suppressing agglomeration due to moisture.

The description regarding the above-described embodiment of the hexagonal boron nitride powder can also be applied to the method for producing a hexagonal boron nitride powder. The method for producing a hexagonal boron nitride powder is not limited to the above-described embodiment. For example, after the firing step, a crushing step of crushing a hexagonal boron nitride powder using a homogenizer applying ultrasound vibration may be performed.

Some embodiments of the present disclosure have been described above, but the present disclosure is not limited to any of the above-described embodiments.

### EXAMPLES

The contents of the present disclosure will be described in more detail with reference to examples and a comparative example, but the present disclosure is not limited to the following examples.

### Example 1

### [Preparation of Hexagonal Boron Nitride Powder]

### <Calcination Step>

100.0 g of a boric acid powder (purity of 99.8 mass % or more, manufactured by Kanto Chemical Co., Inc.) and 90.0 g of a melamine powder (purity of 99.0 mass % or more, manufactured by Wako Pure Chemical Industries, Ltd.) were mixed with each other with an alumina mortar for 10 minutes to obtain a mixed raw material. The mixed raw material after drying was placed in a container made of hexagonal boron nitride and placed in an electric furnace. The temperature was raised from room temperature to 1000° C. at a rate of 10° C./min while circulating nitrogen gas in the electric furnace. After holding the temperature at 1000° C. for 2 hours, the heating was stopped and the mixture was allowed to cool naturally. The electric furnace was open at a point in time when the temperature became 100° C. or lower. In this manner, a calcined product containing low crystalline hexagonal boron nitride was obtained.

### <Firing Step>

3.0 g of sodium carbonate (purity of 99.5 mass % or more) was added to 100.0 g of the calcined product and mixed with each other with an alumina mortar for 10 minutes. The mixture was placed in the above-described electric furnace. The temperature was raised from room temperature to 2000° C. at a rate of 10° C./min while circulating nitrogen gas in the electric furnace. After holding the temperature at 2000° C. for 20 hours, the heating was stopped and the mixture was allowed to cool naturally. The electric furnace was open at a point in time when the temperature became 100° C. or lower. The obtained fired product was collected and pulverized with alumina mortar for 3 minutes to obtain a coarse powder of hexagonal boron nitride.

### <Purification Step>

In order to remove impurities contained in a coarse powder of hexagonal boron nitride, 30 g of the coarse powder was put into 500 g (concentration of nitric acid: 5 mass %) of dilute nitric acid and stirred at room temperature for 60 minutes. After stirring, solid-liquid separation was performed through suction filtration, and washing was performed by replacing the liquid with water (electric conductivity of 1 mS/m) until the filtrate became neutral. After washing, the solid was dried with a dryer at 120° C. for 24 hours to obtain a dry powder. The obtained dry powder was used as a hexagonal boron nitride powder of Example 1.

### [Evaluation of Hexagonal Boron Nitride Powder]

### <Evaluation of Appearance>

The appearance of the obtained hexagonal boron nitride powder was observed. As a result, it was confirmed that the hexagonal boron nitride powder did not agglomerate and had excellent flowability.

### <Evaluation of Charge Attenuation>

The charge amount of the hexagonal boron nitride powder produced in Example 1 was measured with a powder friction charge amount measurement device (manufactured by Nano Seeds Corporation, product name: NS-K100). Specifically, 10 g of a hexagonal boron nitride powder was placed in a PET container with an inner diameter of 90 mm and a height of 120 mm. PEFE stirring blades were placed in the layer of the hexagonal boron nitride powder as shown in FIG. 1 (h=5 mm). Then, the powder was stirred for 5 minutes at a rotation frequency of 300 rpm. As the stirrer, a HEDON three-one motor general-purpose stirrer was used. One-stage stirring vane (“PTEF 4-Blade Screw Stirring Rod” manufactured by SANPLATEC CORPORATION, model number: 23707, rod diameter: 9.5 mm, length: 650 mm, rotary blade diameter (diameter d): 60 mm) having four polytetrafluoroethylene blades was attached to the shaft of this stirrer for stirring. The charge amount of the hexagonal boron nitride powder immediately after stirring was measured with the above-described powder friction charge amount measurement device. The measurement results for the charge amount are as shown in Table 2.

### <Evaluation for Spreadability>

0.2 g of the hexagonal boron nitride powder was placed on one end of an artificial skin (length×width=10 mm×50 mm). The hexagonal boron nitride powder was spread along the surface of the artificial skin in the vertical direction using a spatula so that the hexagonal boron nitride powder was applied to the surface. Image analysis was performed using commercially available image analysis software (WinROOF) to determine the ratio of the coating area of the hexagonal boron nitride powder to the total area of the artificial skin. The larger this area proportion, the better the spreadability. The evaluation criteria for spreadability were as shown in Table 1 depending on the area proportion. The evaluation results for spreadability are as shown in Table 2.

### Example 2

A hexagonal boron nitride powder was prepared in the same manner as in Example 1 except that the holding time in the firing step was set to 25 hours. Then, the hexagonal boron nitride powder was evaluated in the same manner as in Example 1. The evaluation results are as shown in Table 2. The appearance of the obtained hexagonal boron nitride powder was observed. As a result, it was confirmed that the hexagonal boron nitride powder hardly agglomerated and had excellent flowability.

### Example 3

A hexagonal boron nitride powder was prepared in the same manner as in Example 1 except that the holding temperature in the firing step was set to 1950° C. Then, the hexagonal boron nitride powder was evaluated in the same manner as in Example 1. The evaluation results are as shown in Table 2. The appearance of the obtained hexagonal boron nitride powder was observed. As a result, it was confirmed that the hexagonal boron nitride powder hardly agglomerated and had excellent flowability.

### Comparative Example 1

A hexagonal boron nitride powder was prepared in the same manner as in Example 1 except that the firing temperature in the firing step was set to 1700° C. The powder was evaluated in the same manner as in Example 1. The results are as shown in Table 2. The appearance of the obtained hexagonal boron nitride powder was observed. As a result, the hexagonal boron nitride powder agglomerated.

TABLE 1

| Area proportion | Determination |
|---|---|
| 95% or more | Very good |
| 80% or more and less than 95% | Good |
| 60% or more and less than 80% | Fair |
| 40% or more and less than 60% | Poor |
| Less than 40% | Very poor |

TABLE 2

| | Charge amount | Spreadability |
|---|---|---|
| | [nC/g] | [%] |
| Example 1 | −0.50 | Very good |
| Example 2 | −0.10 | Very good |
| Example 3 | 0.55 | Good |
| Comparative Example 1 | 1.10 | Very poor |

The hexagonal boron nitride powders of Examples 1 to 3 had an absolute value of a charge amount of 0.7 nc/g or less. When observing the appearance, Comparative Example 1 formed agglomerated lumps, whereas Examples 1 to 3 clearly had fewer agglomerated lumps than Comparative Example 1. In addition, Examples 1 to 3 had better spreadability than Comparative Example 1.

### INDUSTRIAL APPLICABILITY

According to the present disclosure, it is possible to provide a hexagonal boron nitride powder in which agglomeration due to static electricity is suppressed, and a method for producing the same. In addition, a cosmetic preparation which has excellent spreadability and in which agglomeration is suppressed using the above-described hexagonal boron nitride powder is provided.

### REFERENCE SIGNS LIST

10: Container, 20: Stirrer, 22: Shaft, 24: Stirring vane, 26 Blades, 30: Hexagonal boron nitride powder, 100: Stirring device.

1. A hexagonal boron nitride powder,
wherein an absolute value of a charge amount when 10 g of the hexagonal boron nitride powder is placed in a polyethylene terephthalate container with an inner diameter of 90 mm and a height of 120 mm and stirred at 300 rpm for 5 minutes using a stirring vane 60 mm in diameter with four polytetrafluoroethylene blades is 0.7 nc/g or less.

2. The hexagonal boron nitride powder according to claim 1,
wherein the charge amount is less than −0.1 nc/g.

3. The hexagonal boron nitride powder according to claim 1, which is for a raw material for a cosmetic preparation.

4. A method for producing a hexagonal boron nitride powder, the method comprising:
a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride; and
a firing step of heating and firing a mixed powder containing the calcined product and an aid at 1900° C. to 2100° C. for 10 to 50 hours in an atmosphere of an inert gas, ammonia gas, or a mixed gas thereof.

5. The method for producing a hexagonal boron nitride powder according to claim 4,
wherein a fired product obtained in the firing step is pulverized, washed, and dried to obtain a hexagonal boron nitride powder of which an absolute value of a charge amount when 10 g of the hexagonal boron nitride powder is placed in a polyethylene terephthalate container with an inner diameter of 90 mm and a height of 120 mm and stirred at 300 rpm for 5 minutes using a stirring vane 60 mm in diameter with four polytetrafluoroethylene blades is 0.7 nc/g or less.

6. The method for producing a hexagonal boron nitride powder according to claim 5,
wherein the charge amount is less than −0.1 nc/g.

7. A cosmetic preparation comprising:
the hexagonal boron nitride powder according to claim 1.

8. A method for producing a cosmetic preparation, the method comprising:
producing the cosmetic preparation using the hexagonal boron nitride powder obtained through the method for producing according to claim 4 as a raw material.
